(19) United States
(12) Patent Application Publication
(10) Pub. No.: US 2025/0177400 A1
(43) Pub. Date: Jun. 5, 2025

(54) METHODS OF THERAPEUTICS DEVELOPMENT OF GLIOBLASTOMA MULTIFORME BY QUINAZOLINE DERIVATIVES AND THEREOF

(71) Applicant: Tapan Kumar Khan, Morgantown, WV (US)

(72) Inventor: Tapan Kumar Khan, Morgantown, WV (US)

(21) Appl. No.: 18/526,065

(22) Filed: Dec. 1, 2023

(51) Int. Cl. A61K 31/519; A61K 47/68; A61P 35/00

(52) U.S. Cl. CPC A61K 31/519; A61K 47/6801; A61K 47/6849; A61P 35/00

(57) ABSTRACT

The present invention relates to treating Glioblastoma Multiforme (GBM) patients by reducing cell invasion and cell death using quinazoline derivatives directly inducing apoptosis or through antibody drug conjugation (ADC), anti-TNFRSF19-quinazoline derivatives conjugation. TNFRSF19 (also known as TAJ, TROY, TRADE, and TAJ-alpha) has been identified as a susceptible gene for cancer. It belongs to the TNF receptor superfamily without a known ligand. It increases the survival rate of glioblastoma cells. TNFRSF19 is associated with nasopharyngeal cancer carcinoma, lung cancer risks, and bladder cancer. It can also be used as a biomarker for the above cancers.

### FIELD OF INVENTION

The present invention relates to the method of treating Glioblastoma Multiforme brain tumor and diagnosing nasopharyngeal cancer carcinoma, lung cancer risks, and bladder cancer.

### BACKGROUND OF THE INVENTION

Glioblastoma multiforme (GBM) is a common and most malignant (stage

IV brain tumor) among all brain tumors (˜50% of brain tumors). The median survival rate is ˜14.5 months with no disease-modifying curative treatment. The Standard of Care is radiation, and adjuvant temozolomide (TMZ) chemotherapy after surgical resection is the first-line treatment of GBM. The mechanism of action TMZ is damaging GBM cells by DNA methylation and triggering the death of GBM cells.

Common side effects of TMZ are bone marrow suppression, genotoxic, and teratogenic. DNA damage is not specific to GBM cells; as a result, it is genotoxic. Studies found that 60-75% of cases have no benefit from TMZ chemotherapy (Chamberlain, 2010) with overall survival of 14.6-16.1 months and progression-free survival at 6 months is only 54% of cases. FDA-approved immunotherapeutic agent

Bevacizumab (Avaztin, Genentech) produced some improvement, including cognitive benefit (Kienast et al., 2010), but 40% of patients developed therapeutic resistance in phase II clinical trial (Vredenburgh et al., 2007). Bevacizumab targets therapeutic antibody that binds and inhibits EGF protein in tumor cells. The most common side effects are hypertension (5.5-11.4%), thromboembolic events (3.2-11.9%), gastrointestinal perforation (1.5-5.4%), cerebral bleeding (2-5.3%), wound healing complications (0.8-3.3%), and proteinuria (2.7-11.4%) with overall survival 9.3 months and progression-free survival at 6 months is only 36% cases.

Heterocyclic compounds such as quinazoline scaffolds have enormous potential in modern medicinal chemistry as anti-cancer therapy. Depending on their chemical structures quinazolines act as inducers and inhibitors of apoptosis (Mehndirratta et al., 2016). Apoptosis-inducing quinazolines have potential as anti-tumorogenic agents. Cytotoxicity and genotoxicity of quinazolines are significant problems in cancer therapy.

Quinazoline analogs have been tested for anti-cancer activity for a long time (US 20090036474A1; U.S. Pat. No. 5,457,105A). Quinazoline derivatives and conjugates help inhibit the growth of brain tumor cells through human epidermal growth factor (EGF) and CDK5 (WO1999061428A1; Peyressatre et al., 2020). Recent patents on the anti-cancer effect of Quinazoline were reviewed (Raveg et al., 2015).

Here we report the discovery and optimization of synthetic quinazoline derivatives that inhibited glioblastoma cell invasion and survival. We have developed synthetic agents that induce GBM cell death by reducing cellular adhesive proteins such as β-integrin and reducing oncogenic protein kinase C (PKC-epsilon) activity. One of the leading quinazoline derivatives was non-toxic to human primary skin fibroblasts.

In cell adhesion dynamics, integrins and extracellular matrix (ECM) mediate interactions required for tumor cell invasion and metastasis. Since the interaction of integrin β-1 and ECM protects glioma cells drug-induced anoikis (Uhm et al., 1999) targeting integrin β1 has potential as antiangiogenic therapy for glioblastoma (Carbonell et al., 2013).

Targeting TNFRSF19 (TROY) for therapeutic development for glioblastoma treatment is based on the fact that TNFRSF19 is highly expressed in glioma tumors and at the site of GBM cell invasion (Paulino et al., 2010), silencing TNFRSF19 by siRNA inhibits glioblastoma cell invasion (Loftus et al., 2013), and the expression levels of TNFRSF19 are proportional to the glial tumor grade and the resistance to temozolomide (TMZ) and radiation therapy (Ding et al., 2017). TNFRSF19 has been identified as a susceptible gene for cancer. Besides GBM, TNFRSF19 is associated with the pathological stages of bladder cancer patients (Nomura et al., 2019). TNFRSF19 is a genetic susceptibility gene in nasopharyngeal carcinoma and other cancers (Dai et al. 2016). targeting TROY inhibitors will be specific to other dreadful cancers.

### BRIEF DESCRIPTION OF THE DRAWINGS

FIG. 1 depicts the chemical structures of synthetic quinazoline derivatives.
1. 6-Pyridin-2-yl-5,6-dihydro-benzo[4,5]imidazo[1,2-c]quinazoline
2. 6-Pyridin-2-yl-benzo[4,5]imidazo[1,2-c]quinazoline
3. 5,6,5′,6′-Tetrahydro-[6,6′]bi[benzo[4,5]imidazo[1,2-c]quinazolinyl]
4. [6,6′]Bi[benzo[4,5]imidazo[1,2-c]quinazolinyl]
5. Benzo[4,5]imidazo[1,2-c]quinazolin-6-yl-methanol
6. 6-(1H-Pyrrol-2-yl)-5,6-dihydro-benzo[4,5]imidazo[1,2-c]quinazoline
7. 6-(1H-Pyrrol-2-yl)-benzo[4,5]imidazo[1,2-c]quinazoline
8. 6-Methyl-5,6-dihydro-benzo[4,5]imidazo[1,2-c]quinazoline
9. 6-Thiophen-2-yl-5,6-dihydro-benzo[4,5]imidazo[1,2-c]quinazoline
10. 6-Thiophen-2-yl-benzo[4,5]imidazo[1,2-c]quinazoline
11. (5,6-Dihydro-benzo[4,5]imidazo[1,2-c]quinazolin-6-yl)-ethanone

FIG. 2 depicts a novel quinazoline derivative (Compound 1:6-Pyridin-2-yl -5,6-dihydro-benzo[4,5]imidazo[1,2-c]quinazoline) that induces apoptosis in Glioblastoma (GBM) cells. Cells were treated with different concentrations of Compound 1 (0.1, 1, 10, 50, 100μ) or vehicle (control). After three days under treatment at 50 and 100 μM (FIG. 2E, F) concentrations, Compound 1 induces significant apoptosis compared to control (2A).

FIG. 3 depicts concentration dependence cell viability of human glioblastoma multiforme (GBM) cells by novel quinazoline derivative (Compound 1: 6-Pyridin-2-yl-5,6-dihydro-benzo[4,5]imidazo[1,2-c]quinazoline). Compound 1 treatments (3 days) induce apoptosis in 50 μM and 100 μM concentrations.

FIG. 4 depicts a comparative intervention of induction of cellular apoptosis in GBM cells by novel quinazoline derivative (Compound 1) and Propentofylline. Compound 1 is more potent for inhibiting GBM survival than Propentofylline. Propentofylline is a xanthine derivative that inhibits phosphodiesterase.

FIG. 5 depicts the Human glioblastoma multiforme (GBM) cell invasion in a three-dimensional Matrigel basement membrane. Model Tumor Spheroid assay: GBM cells form a spheroid tumor-like biological entity in three-dimensional space. Proliferation and invasion of GBM in 3-dimensional Matrigel matrix in 5 days (A) and 12 days (B). Arrows indicate invasive cells in model tumor spheroids. The proliferation and invasion of GBM in a three-dimensional Matrigel matrix have been studied for days. On the 5th day, there were markedly differences in tumor size and number of invasive cells by treatment with the most potent quinazoline compound (A), control (Vehicle) (B), and Propentofylline (C). Arrows indicate invasive cells. Compound 1 is more potent than Propentofylline in terms of reducing tumor size and cell invasion.

FIG. 6 depicts a novel quinazoline derivative (Compound 1:-Pyridin-2-yl -5,6-dihydro-benzo[4,5]imidazo[1,2-c]quinazoline) that decreases glioblastoma cell invasion by decreasing integrin β1 (CD29) levels. Intergrin β1 band decreased in Western blot analyses both in normal culture (A), and serum-free condition (B) by the treatment of compound 1. Propentofylline treatment produces reverse effects (A). Relative expression levels of integrin β1 is normalized with β-tubulin as a loading control in Western blot analyses. Compound 1 significantly decreases integrin β1 expression level (C), whereas propentofylline significantly increases integrin β1 expression levels (D) at similar concentrations. Standard Error of Mean (SEM) is calculated from three independent measurements.

FIG. 7 depicts how novel quinazoline derivative decreases oncogenic PKCε activity in neuroblastoma cells. C=concentration of PKCε in cytosol fraction, and M=concentration of PKCε in membrane fraction of cells. PKCε activity was measured as the ratio of the concentration in the membrane and cytosol fraction.

FIG. 8 depicts the non-toxicity of Compound 1. There is no noticeable cell death when cultured human normal primary skin fibroblasts isolated from punch biopsies are treated with Compound 1.

FIG. 9 depicts the scheme for the antibody-drug-conjugation.

### DETAILED DESCRIPTION OF THE INVENTION

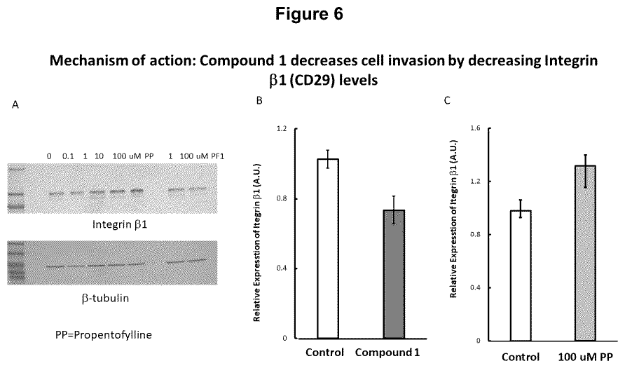

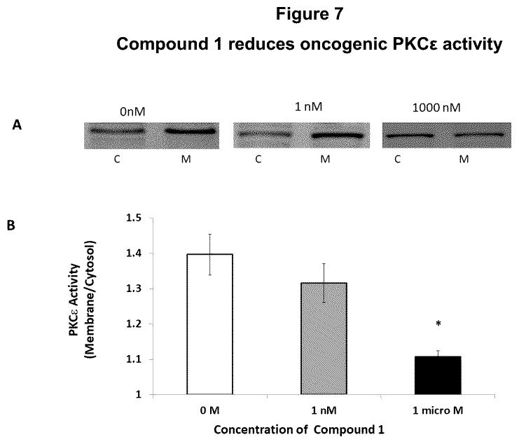

The Invention provides methods for inhibition of glioblastoma cells by synthetic quinazoline derivatives as a new therapeutic approach. Novel quinazoline derivatives induced glioblastoma cell death and decreased glioblastoma cell invasion. This specific synthetic compound induced GBM cell death stopped the cellular invasion in the three-dimensional Matrigel matrix and was highly specific to GBM cell death when tested with other cells. Some of the potent synthetic quinazoline derivatives are non-toxic to normal non-tumorigenic cells but toxic to cancerous cells. Newly discovered novel quinazoline derivatives can be synthesized very easily (less than 4 reaction steps) with very high yields. The most potent quinazoline derivative (6-Pyridin-2-yl-5,6-dihydro-benzo[4,5]imidazo[1,2-c]quinazoline) was synthesized by one chemical step with 90% yield (FIG. 1). Pharmacological intervention to GBM cells by the most potent quinazoline derivative caused increased GBM cell apoptosis and decreased GBM cell invasion by inhibiting beta-1 integrin (FIG. 6). In cell adhesion dynamics integrins and extracellular matrix (ECM) mediate interactions that are required for tumor cell invasion and metastasis. Since the interaction of integrin β1 and ECM protects glioma cells, drug-induced anoikis targeting integrin β1 has potential as an antiangiogenic therapy for glioblastoma. Novel quinazoline derivative decreases oncogenic PKC-epsilon activity in neuroblastoma cells (FIG. 7). Most potent synthetic quinazoline derivatives can be used to effectively treat GBM alone or combined with other chemotherapeutic/immunotherapeutic agents.

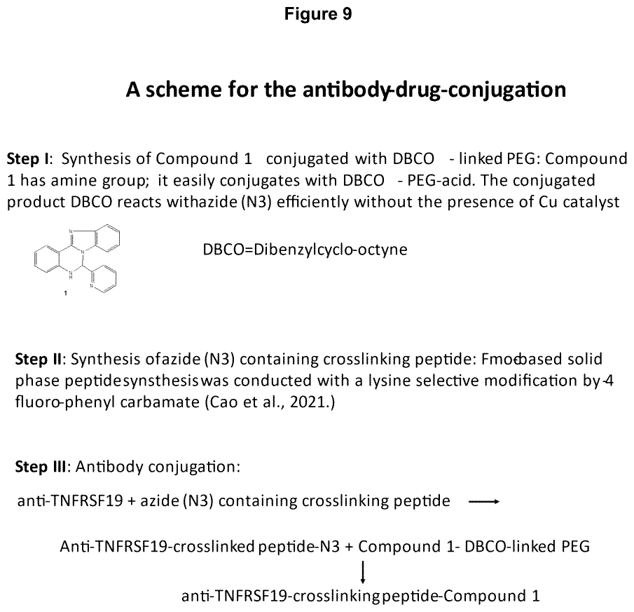

In addition, the invention schemed a method of antibody-conjugated therapy for GBM using anti-TNFRSF19 (TROY). Most of the GBM cells express TNFRSF19 on their cell surface. When anti-TROY conjugated with Quinazoline derivatives should lower amount of Quinazoline required to induce apoptosis in GBM cells (FIG. 9). Antibody conjugation may reduce the cytotoxicity and more targeted delivery, like “Durg Missile”. Quinazoline derivatives in the conjugated form will remain stable in blood for a longer time and hit accurately to the targeted cells.

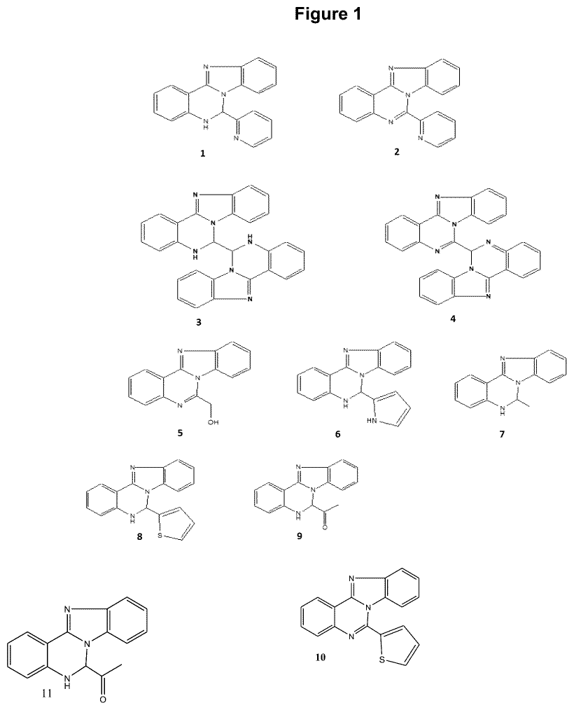

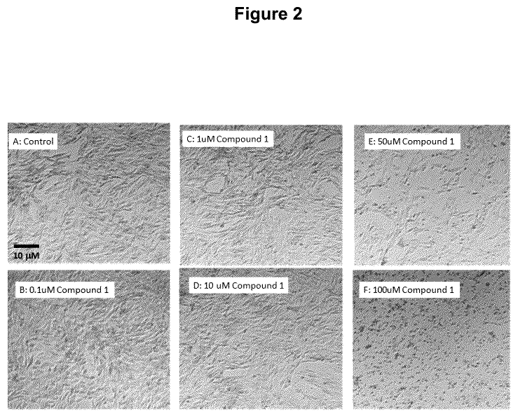

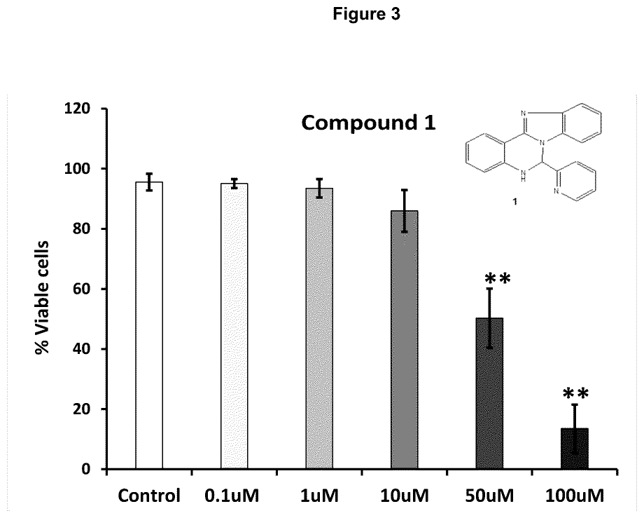

Glioblastoma multiforme (GBM) cells (T98-G) were cultured in 6 wells for ˜90% confluent. Cells were treated with different concentrations of Compound 1 (0.1, 1, 10, 50, 100μ) or vehicle (control). After three days under treatment at 50 and 100 μM (FIG. 2E, F) concentrations Compound 1 induced significant apoptosis compared to control (2A). Cell viability was tested by trypan blue exclusion assay. Cell viability was reduced by apoptosis at treatment of 50 and 100 μM concentrations Compound 1 (FIG. 3).

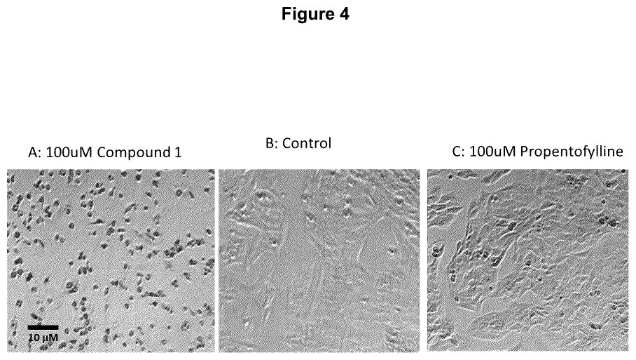

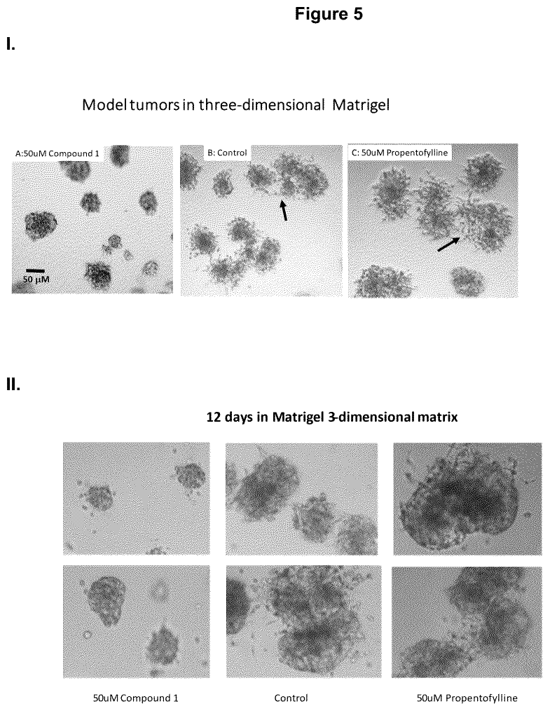

Novel quinazoline derivative Compound 1 decreased GBM cell invasion in a 3-dimensional Matrigel matrix (FIG. 5). GBM cells were incubated in a previously prepared 3-dimensional Matrigel matrix. Multicellular spheroids were formed after three days of incubation. The mechanical properties of cell invasion of patient-derived primary glioblastoma cells have been studied recently in 3-D matrigel matric (Grundy et al., 2016). Cellular invasiveness was monitored in multicellular spheroids of GBM embedded in 3D matrigel matric (FIG. 5). Invasive cells were sprouting outside of the spheroid in the case of control (FIG. 5B) and 50 μM Propentopfylline (FIG. 4C) treatment. There were no invasive cells when cells were treated with 50 μM Compound 1 (FIG. 5A). Several studies showed that propentofylline decreased GBM cell invasion (Dhruv et al., 2016; Jacob et al., 2012). Our invention found Compound 1 was a more potent inhibitor of GBM cell invasion (FIG. 4A).

Novel quinazoline derivative Compound 1 is more potent than Propentofylline to induce apoptosis in GBM cells (FIG. 4). Previous studies showed that propentofylline inhibits GBM survival (Dhruv et al., 2016). Our invention found that the novel quinazoline derivative Compound 1 is more potent than Propentofylline in terms of inducing apoptosis in GBM cells (FIG. 4).

Novel quinazoline derivative (Compound 1) decreased cell invasion by decreasing β1 integrin (CD29) levels (FIG. 6). Integrins have an important role to play in cell invasion in tumorigenic environments. They are receptors of various ECM components. β1 integrin was found to be the main component of the signaling pathway stimulating glioma cell invasion (Paulus et al., 1996). Moreover, the role of β1 integrin has been implicated in tumor vascularization both by VEGF-dependent and VEGF-independent manner of promoting endothelial cell migration (Leenders et al., 2004; Azam et al., 2010). β1 integrin levels were upregulated in GBM cells that acquired resistance to bevacizumab (Avastin; Genentech) therapy (Carbonell et al., 2013) We intend to find the capability of the most potent quinazoline derivative to reduce β1 integrin level. The most potent quinazoline derivative decreased levels of β1 integrin level significantly. We also compared the effectiveness of reducing β1 integrin level with propentofylline in the same concentration. Propentofylline was not able to reduce β1 integrin level. Deletion of β1 integrin reduced tumorigenesis in in-vivo xenograft tumor growth animal model (Hou et al., 2016).

Novel quinazoline derivative decreases oncogenic PKCepsilon (PKCε) activity in neuroblastoma cells (FIG. 7). We tested whether novel quinazoline derivatives can decrease oncogenic PKCε activity. Compound 1 significantly decreased the PKCε activity in SHSY5Y cells (FIG. 7). Oncogenic PKCε is a survival kinase for GBM cells (Okhrimentko et al., 2005).

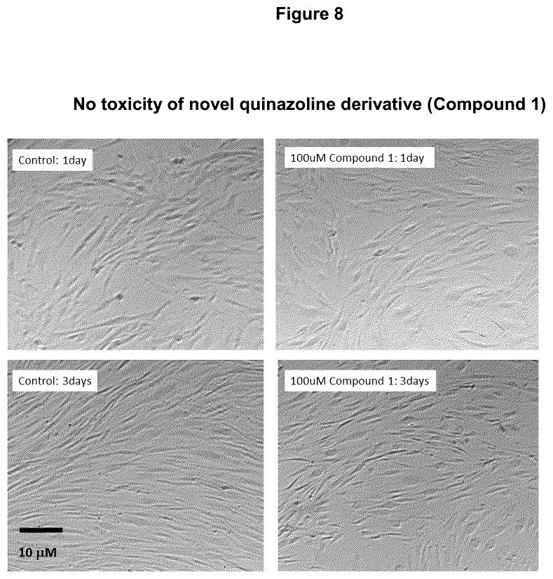

Insignificant toxicity was found for the quinazoline derivative (Compound 1): One quinazoline derivative (Compound 1) was non-toxic to cultured normal primary skin fibroblasts isolated from healthy individuals (FIG. 8). After three days of continuous treatment by 100 μM Compound 1, there was no cell death. Compound 1 was found to be non-toxic to primary human skin fibroblast cell lines.

### Definitions

Glioblastoma Multiforme refers to a most common and fast-growing tumor in the brain and spinal. It contains highly variable glial cells. Multiforme is used to mean the multiple types of cells.

Quinazolines refer to heterocyclic compounds consisting of at least two rings, a benzene ring fused with a pyrimidine ring. Quinazolinones and quinazolines are of importance in medicinal chemistry. Those have various medicinal properties like antibacterial, antifungal, anti-inflammatory, and anti-cancer.

Propentofylline refers to a xanthine derivative compound and phosphodiesterase inhibitor with neuroprotective properties and the ability to cross blood-brain barriers. It has been studied for Alzheimer's and multi-infarct dementia and is a possible adjunct in treating ischemic stroke without much success (Frampton et al., 2003). It works for dementia cases in two possible mechanisms: it reduces oxidative stress by reducing the production of free radicals and reduces inflammation by deactivating microglial cells.

Antibody-drug-conjugation (ADC) refers to a drug conjugated to an antibody by a chemical linker. ADC is used to take advantage of highly specific binding to a particular cancer cell that expresses a specific antigen and is highly potent to kill the cancer cells by the drug. ADC effectively reduces the toxicity of toxic drugs by reducing the binding to normal cells. It improves the effectiveness of chemotherapeutic agents.

PKCε refers to one of the ten Protein kinase C (PKC) isoforms. There are three subtypes of PKC isozymes, e.g., classical isoforms (α, βI, βII, and γ), the novel isoforms (δ, ε, θ, and η), and atypical isoforms (ζ, and I/λ). PKCε belongs to the second group, which does not require calcium for their activation. PKCε acts as a transforming oncogene and plays an important role in establishing an aggressive metastatic phenotype of tumor and cancer. Overexpression of PKCε is associated with poor treatment response in metastatic cancers. PKCε is overexpressed in GBM cell cultures. PKCε activates ERK to mediate integrin-dependent glioma cell adhesion and motility in GBM.

Integrin β1 refers to a focal adhesion protein highly expressed in cancer cells' extracellular matrix. It belongs to a large family of αβ heterodimeric transmembrane proteins. A better adhesion increases the survival of cancer cells and association with other surrounding cells. As a result, it helps their migratory behavior and increases metastatic capabilities. Integrins, including β1 integrin, represent therapeutic targets for tumors and cancer because expression level modifications in tumor cells can be a target for modulating surrounding cancer-associated cells, tumor initiation, and progression.

TNFRSF19 refers to a transmembrane TNF family receptor. It is also known as TAJ, TROY, TRADE, and TAJ-alpha. It has been identified as a susceptible gene for cancer. Like p75NTR, TNFRSF-19 (TNFSF member 19) expresses in developing and adult brain and mediates NF-kB activation via TRAF. It also acts as a co-receptor in the myelin-inhibitory protein complex that inhibits axonal regeneration.

Primary skin fibroblasts refer to fibroblast cells isolated and cultured from skin biopsies of dermal layers. Those cells proliferate for several passages, keeping their primary cell behavior.

Matrigel matrix refers to a basement membrane secreted by Engelbreth-Holm-Swarm (EHS) mouse sarcoma cells. It contains laminin, nidogen, collagen, and heparan sulfate proteoglycans, growth factors like TGF-beta and EGF that prevent differentiation and induce the proliferation of many cell types. Three-dimensional Matrigel matrix is used to culture tumor cells as the model for metastasis model and migrating cells are used as tumor models.

### Description of Embodiments

In one embodiment, specific quinazoline compounds inhibit GBM inhibition and reduce tumor progression in three-dimensional tumor models. In certain embodiments, specific quinazoline compounds induce apoptosis GBM and cure GBM.

In another embodiment, specific quinazoline compounds conjugate with TROY-antibody, wherein antibody-drug-conjugation (ADC) works as a “biological missile” for a pointed target in GMB.

In certain embodiments, we develop ACD specific to cell surface TNFRSF19 that binds with glioblastoma cells and kills/induces apoptosis in those cells. Targeted antibodies specifically bind with Glioblastoma cells expressing TROY, and conjugated anti-cancer drugs kill cancerous cells. Finally, the antibody-drug conjugate will be used for clinical development.

In certain embodiments, synthetic Quinazoline derivatives are more potent than Propentofylline in terms of inducing GBM cell invasion. Propentofylline (molecular weight 306.366) is a synthetic methyl xanthine derivative recently shown to be decreasing GBM cell invasion and survival (Dhruv et al., 2016). However, it was found to be ineffective inducing apoptosis in GBM cells (Jacobs et al., 2012). In the present invention, we compare the ability to induce apoptosis and reduce cellular invasion of GBM by novel synthetic derivatives of quinazoline with propentofylline. Propentofylline did not induce apoptosis in GBM cells in culture conditions. At the same time, two quinazoline derivatives (Compound 1 and Compound 7) caused significant apoptosis in GBM cells at 50-100 μM concentrations.

In certain embodiments, Quinazoline derivatives reduce the expression levels of β1 integrin. Integrins are a class of molecules involved in GBM cell adhesion, migration, and cellular invasion (Paolillo et al., 2016). These transmembrane receptors facilitate extracellular matrix adhesion by binding them with glycoproteins such as laminins and collagens. β1 integrin regulates adhesion, migration, and invasion of all kinds of cancer cells. Integrin also increases the chances of survival of malignant cells by increasing anti-apoptotic proteins such as bcl-2 and FLIP. Here, we demonstrated that Compound 1 significantly reduced expression levels of β1-Integrin, whereas propentofylline increased expression levels of β1-Integrin in GBM cells at similar concentrations.

In a further embodiment, TROY has no ligand discovered. Here, we design to inhibit the inhibition of glioblastoma cells by targeting TROY. TROY is highly expressed in glioblastoma cell lines.

### Biological Samples

Cell lines and culture conditions: Human glioblastoma cells. The human

GBM cell line T98-G (ATCC-CRL-1690) was obtained and authenticated by the American Type Culture Collection (Cat #30-2003; ATCC, Manassas, VA). Cells were cultured and maintained in low glucose Dulbecco's Modified Eagle Medium (DMEM) (Thermo Fisher Scientific) supplemented with 10% fetal bovine serum (FBS, Thermo Fisher Scientific) at 37° C. in a 5% CO2 humidified atmosphere and passed in less than 6 months. Human neuroblastoma cells. Human SH-SY5Y neuroblastoma cells (Sigma-Aldrich) were cultured in 45% F12K, 45% Modified Eagle Medium (MEM), supplemented with 10% FBS in tissue culture incubator (37° C., 5% CO2, 90% humidity). Primary skin fibroblasts. Primary skin fibroblasts were obtained from the Coriell Institute for Medical Research (Camden, NJ; Cat #AG09555; the Aging Cell Culture Repository of the National Institute on Aging) and maintained in DMEM with low glucose (Invitrogen, Grand Island, NY) supplemented with 10% FBS in 6-well culture plates (37° C., 5% CO2, 90% humidity) until reaching 90-100% confluence. In addition, cells were treated with either vehicle, 50, and 100 μM quinazoline derivatives and propentofylline. The condition of the cultured cells was monitored with an inverted cell culture microscope (Westover Digital AMID Model 2000, Westover Scientific, Bothell, WA), controlled by a computer, and images were captured with image acquisition software (Micron 2.0.0, Westover Scientific).

Reagents and antibodies: Propentofylline (molecular weight 306.366; [1-(5O-oxohexyl)-3-methyl-7-propylxanthine) was purchased from Sigma-Aldrich (St. Louis). Propentofylline and synthetic quinazoline derivatives were dissolved in DMSO (30-60 mM, depending on solubility). Aliquots were stored at −20 C. Stock solutions were dissolved in a culture medium at different concentrations just before each experiment. Rabbit monoclonal anti-Integrin beta 1 [CD29] antibody (Cat #04-1109) was purchased from Millipore (Temecula, CA). Anti-tubulin III antibody (Cat #79-720) was purchased from ProSci (Poway, CA). Antibody for PKCε (C-15) (Santa Cruz Biotechnology, Santa Cruz, CA; Cat No: sc-214) was used to detect PKCε and has been recommended for detection of PKCε of human origin by western blotting and immunofluorescence analysis by the manufacturer. Quinazoline derivatives were synthesized in the laboratory.

### Example 1

### Synthesis: Compound 1

Preparation of [6-(2-pyridinyl)-5,6-dihydrobenzimidazo[1,2-c]quinazoline] Method: Ethanolic solution of 2-(2-aminophenyl) benzimidazole, (2.09 g, 10.0 mmol) was added to pyridine-2-carboxylaldehyde (1.07 g, 10.0 mmol) in ethanol (25 mL) at room temperature. After refluxing for 4 h in ethanol, yellow colored solution was obtained. The compound was precipitated as a white-colored crystalline through slow evaporation of the solvent.

1. A novel therapeutic approach, quinazoline derivatives induced glioblastoma cell death and decreased glioblastoma cell invasion that can be used as a replacement of TMZ.

2. The method of claim 1, wherein said, these specific non-toxic synthetic compounds can be used for GBM cell death and to stop the cellular invasion in the three-dimensional Matrigel matrix.

3. The method of claim 1, wherein said, some potent synthetic quinazoline derivatives are non-toxic to normal non-tumorigenic cells but toxic to cancerous cells.

4. The method of claim 1, wherein said, novel quinazoline derivatives can easily be synthesized (less than 4 reaction steps) with very high yields.

5. In claim 1, one of the most potent quinazoline derivative (6-Pyridin-2-yl-5,6-dihydro-benzo [4,5]imidazo[1,2-c]quinazoline) was synthesized by one chemical step with 90% yield.

6. The invention of claim 1 further comprising pharmacological intervention to GBM cells by the most potent quinazoline derivatives caused increased GBM cell apoptosis and decreased GBM cell invasion by inhibiting beta-1 integrin.

7. The invention of claim 1 further comprising specific quinazoline derivatives decreases oncogenic PKC-epsilon activity in neuroblastoma cells.

8. The invention of claim 1 further comprising that potent synthetic quinazoline derivatives can be used to effectively treat GBM alone or combined with other chemotherapeutic/immunotherapeutic agents.

9. Anti-TNFRSF-19 (TROY) conjugated with quinazoline derivatives is very potent in inducing apoptosis.

10. TNFRSF19 (TROY) can be used as a biomarker for numerous cancer patients (e.g., nasopharyngeal cancer carcinoma, lung cancer risks, and bladder cancer).
